(19) United States
(12) Patent Application Publication
(10) Pub. No.: US 2024/0390578 A1
(43) Pub. Date: Nov. 28, 2024

(54) DETACHABLE ACTIVATION MECHANISM AND NEEDLE CARTRIDGE ASSEMBLY FOR INSERTING INFUSION NEEDLE AND CGM SENSOR

(71) Applicant: AITA BIO INC., New York, NY (US)

(72) Inventors: Dilan Casanovas Mack, New York, NY (US); Russell Cole, River Vale, NJ (US)

(21) Appl. No.: 18/772,138

(22) Filed: Jul. 13, 2024

Related U.S. Application Data
(63) Continuation of application No. 18/724,048, filed on Jan. 1, 0001.
(60) Provisional application No. 63/302,205, filed on Jan. 24, 2022.
(60) Provisional application No. 63/415,421, filed on Oct. 12, 2022.

(51) Int. Cl. A61M 5/142; A61M 5/172

(52) U.S. Cl. CPC A61M 5/14248; A61M 5/1723; A61M 2005/14252; A61M 2005/1726; A61M 2230/201

(57) ABSTRACT

An infusion system comprising: a device for delivering insulin to a user, the device configured to be mounted the user; a CGM sensor for measuring glucose level in the user and an infusion needle for delivering the insulin to the user; a needle cartridge assembly for supporting the CGM sensor and/or the infusion needle, the needle cartridge assembly configured to move from (1) a telescoping position above the device for delivering insulin, wherein the CGM sensor and/or infusion needle are/is in a retracted position within the device for delivering insulin to (2) an advanced position within the device, wherein the infusion needle and/or CGM sensor are/is in a deployed position inserted into a subcutaneous tissue of the user's skin; and a detachable activation mechanism for (a) moving the needle cartridge assembly from the telescoping position to the advanced position and for (b) releasing the device for delivering insulin upon activation of the detachable activation by the user.

### CROSS-REFERENCE TO RELATED APPLICATIONS

This is a continuation application of national phase application Ser. No. 18/724,048, filed Jun. 25, 2024, entitled “Detachable Activation Mechanism and Needle Cartridge Assembly for Inserting Infusion Needle and CGM Sensor” which claims priority to PCT application PCT/US23/11318, filed Jan. 23, 2023 which claims priority to U.S. provisional application No. 63/302,205, filed Jan. 24, 2022, entitled “Detachable Activation Mechanism for Inserting An Integrated Infusion Needle and CGM Sensor” and U.S. provisional application No. 63/415,421, filed Oct. 12, 2022 entitled “Cartridge Assembly With Infusion Catheter and CGM Sensor For simultaneous Insertion” which are all incorporated by reference herein.

### FIELD OF THE INVENTION

The present invention relates to a detachable activation mechanism and a needle cartridge assembly for inserting an infusion needle and CGM sensor.

### BACKGROUND OF THE INVENTION

Insulin pumps help people with diabetes to conveniently manage their blood sugar. These devices deliver insulin at specific times. Insulin patch pumps or pods are one type of insulin pump. The pods are wearable devices that adhere to the skin of a user using an adhesive patch. The pods deliver insulin from a chamber and internal cannula based on separately acquired CGM sensor readings. The pods are controlled wirelessly with a handheld controller.

It would be advantageous to provide improvements to insulin pumps described above.

### SUMMARY OF THE INVENTION

A detachable activation mechanism and cartridge assembly for inserting a needle and CGM sensor needle are disclosed.

In accordance with an embodiment of the disclosure, an infusion system comprising: a device for delivering medicament to a user, the device configured to be mounted to the user; an infusion needle for delivering the medicament to the user; a needle cartridge assembly for supporting the infusion needle, the needle cartridge assembly configured to move from (1) a telescoping position above the device for delivering medicament, wherein the infusion needle is in a retracted position within the device for delivering medicament to (2) an advanced position within the device, wherein the infusion needle is in a deployed position inserted into a subcutaneous tissue of the user's skin; and a detachable activation mechanism for (a) moving the needle cartridge assembly from the telescoping position to the advanced position and for (b) releasing the device for delivering medicament upon activation of the detachable activation by the user.

In accordance with another embodiment of the disclosure, an infusion system comprising: a device for delivering medicament to a user, the device configured to be mounted the user, the device including a analyte sensor for measuring an analyte in a user and/or an infusion needle for delivering the medicament to the user; and a detachable activation mechanism for deploying the device on the user and detaching from the device subsequent to deployment, wherein the detachable activation mechanism is configured to move the analyte sensor and/or the infusion needle from (1) a retracted position within the device for delivering medicament wherein the analyte sensor and/or the needle are/is retracted within the device for delivering medicament to (2) an advanced position, wherein the analyte sensor and/or the needle are/is in a deployed position inserted into a subcutaneous tissue of the user's skin.

In accordance with yet another embodiment of the disclosure, an infusion system comprising: a device for delivering medicament to a user, the device configured to be mounted on the user; an infusion needle for delivering the medicament to the user; and a needle cartridge assembly for supporting the infusion needle, the needle cartridge assembly configured to move from (1) a telescoping position above the device for delivering medicament, wherein the infusion needle is in a retracted position within the device for delivering medicament to (2) an advanced position within the device, wherein the infusion needle is in a deployed position inserted into a subcutaneous tissue of the user's skin.

In accordance with yet another embodiment of the disclosure, an infusion system comprising: a device for delivering medicament to a user, the device configured to be mounted the user; an analyte sensor for measuring an analyte in the user and an infusion needle for delivering the medicament to the user; a needle cartridge assembly for supporting the CGM sensor and/or the infusion needle, the needle cartridge assembly configured to move from (1) a telescoping position above the device for delivering medicament, wherein the analyte sensor and/or infusion needle are/is in a retracted position within the device for delivering medicament to (2) an advanced position within the device, wherein the infusion needle and/or analyte sensor are/is in a deployed position inserted into a subcutaneous tissue of the user's skin; and a detachable activation mechanism for (a) moving the needle cartridge assembly from the telescoping position to the advanced position and for (b) releasing the device for delivering medicament upon activation of the detachable activation by the user.

In accordance with another embodiment of the disclosure, an infusion system comprising: a device for delivering medicament to a user, the device configured to be mounted the user; a needle for infusing medicament into the user or introducing an infusion catheter to infuse the medicament into the user; a needle cartridge assembly for supporting the needle, the needle cartridge assembly configured to move from (1) a telescoping position above the device for delivering the medicament, wherein the needle is in a retracted position within the device for delivering the medicament to (2) an advanced position within the device, wherein the needle is in a deployed position inserted into a subcutaneous tissue of the user's skin; and a detachable activation mechanism for (a) moving the needle cartridge assembly from the telescoping position to the advanced position and for (b) releasing the device for delivering medicament upon activation of the detachable activation by the user.

In accordance with another embodiment of the disclosure, an infusion system comprising: a device for delivering medicament to a user, the device configured to be mounted on the user; a needle for infusing medicament into the user or introducing an infusion catheter to infuse the medicament into the user; a needle cartridge assembly for supporting the needle, the needle cartridge assembly configured to move from (1) a telescoping position above the device for delivering medicament, wherein the needle is in a retracted position within the device for delivering medicament to (2) an advanced position within the device, wherein the needle is in a deployed position inserted into a subcutaneous tissue of the user's skin.

In accordance with another embodiment of the disclosure, an infusion system comprising: a device for delivering medicament to a user, the device configured to be mounted to the user, the device including needle for delivering the medicament to the user or introducing a catheter for infusing medicament to the user; and a detachable activation mechanism for deploying the device on the user and detaching from the device subsequent to deployment, wherein the detachable activation mechanism is configured to move the needle from (1) a retracted position within the device for delivering medicament wherein the needle is retracted within the device for delivering medicament to (2) an advanced position, wherein the needle is in a deployed position inserted into a subcutaneous tissue of the user's skin.

In accordance with another embodiment of the disclosure, an infusion system comprising: a device for delivering medicament to a user, the device for delivering configured to be mounted to the user, the device including a first introducer needle for introducing an infusion catheter into the user for infusing the medicament and a second introducer needle for introducing an analyte sensor into the user for sensing an analyte in the user; a cartridge assembly configured to move from (1) a telescoping position above the device, wherein the first and second introducer needles are in a retracted position within the device for delivering medicament and (2) an advanced position within the device, wherein the first and second introducer needles are in a deployed position inserted into tissue of the user; and a detachable activation mechanism for causing the cartridge assembly to move from (1) the telescoping position to (2) the advanced position in the device.

In accordance with another embodiment of the disclosure, an infusion system comprising: a device for delivering medicament to a user, the device for delivering configured to be mounted to the user, the device including a first introducer needle for introducing an infusion catheter into the user for infusing the medicament into the user and a second introducer needle for introducing an analyte sensor into the user for sensing an analyte in the user; and a detachable activation mechanism for deploying the device for delivering on the user and for detaching from the device subsequent to deployment, wherein the detachable activation mechanism is configured to cause the cartridge assembly to move from (1) the telescoping position to (2) the advanced position in the device.

In accordance with another embodiment of the disclosure, an infusion system comprising: a device for delivering medicament to a user, the device for delivering configured to be mounted to the user, the device including a first introducer needle for introducing an infusion catheter into the user for infusing the medicament into the user and a second introducer needle for introducing an analyte sensor into the user for sensing an analyte in the user; and a cartridge assembly supporting the first and second introducer needles, the cartridge assembly configured to move from (1) a telescoping position above the device, wherein the first and second introducer needles are in a retracted position within the device for delivering medicament and (2) an advanced position in the device, wherein the first and second introducer needles are in a deployed position inserted into tissue of the user.

### BRIEF DESCRIPTION OF DRAWINGS

FIGS. 1-2 depict perspective views of an example infusion system for infusing insulin into a user in a pre-activation and post-activation configuration, respectively.

FIG. 3 depicts a perspective view of the infusion system in FIG. 2 wherein a device for delivering insulin is shown without a needle cartridge assembly.

FIGS. 4A-D depict various views of a needle cartridge assembly of the example infusion system of FIGS. 1-2.

FIGS. 5 and 6 depict the needle cartridge assembly of the example infusion system in FIGS. 1 and 2 in a telescoping position.

FIG. 7 depicts a front perspective view of the infusion system in FIG. 2 in which the needle cartridge assembly, in an advanced position, is exposed.

FIG. 8 depicts a cross sectional view (in part) of the infusion system of FIG. 2 in a post activation configuration in which the needle cartridge assembly has fully advanced in the device for delivering insulin of the infusion system.

FIG. 9 depicts a cross sectional view of a baseplate of the infusion system of FIG. 2.

FIG. 10 depicts the components of the infusion system of shown in FIG. 1 in an exploded configuration.

FIGS. 11-13 depict various cross-sectional views of the infusion system of FIG. 1 in a pre-activation configuration.

FIGS. 14-16 depict various cross-sectional views of the infusion system of FIG. 1 in a post activation configuration.

FIG. 17 depicts a block diagram of example components of the device for delivering insulin and needle cartridge assembly of the infusion system.

FIGS. 18 and 19 depict bottom perspective views of an infusion system for infusing insulin into a user in pre-activation and post activation configurations, respectively.

FIG. 20 depicts a view of a cartridge assembly of FIG. 18.

FIG. 21 depicts a view of the cartridge assembly of FIGS. 18 and 19 with introducer needles removed.

FIG. 22 depicts a side view of the cartridge assembly fluidically and electrically connected to the device (or pod) for delivering insulin to the user.

FIG. 23 depicts an exploded view of the cartridge assembly components.

FIG. 24 depicts a view of the portion of the device (or pod) for delivering insulin to the user exposing inner components.

FIG. 25 depicts a view of another example cartridge assembly.

FIG. 26 depicts an exploded view of the cartridge assembly shown in FIG. 25.

FIG. 27 depicts a side view of the cartridge assembly installed within device (or pod) for delivering insulin to the user but before it is assembled into detachable activation mechanism.

FIG. 28 depicts a cross sectional view of an infusion system in which device (or pod) for delivering insulin to the user is in a pre-activation configuration that is assembled into the detachable activation mechanism.

FIG. 29 depicts a transparent view of the infusion system in which device (or pod) for delivering insulin to the user is in a pre-activation configuration with the detachable activation mechanism.

FIGS. 30 and 31 depict views of the device (or pod) for delivering insulin to the user where after activation, the user has removed the detachable activation mechanism.

FIGS. 32 and 33 depict views of the device (or pod) for delivering insulin to the user wherein the introducer needles have been removed using an insertion needle hub.

### DETAILED DESCRIPTION OF THE INVENTION

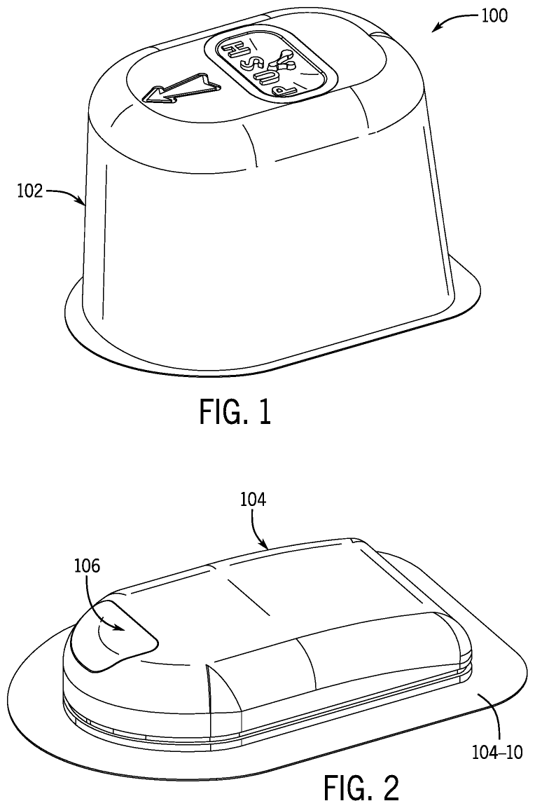

FIGS. 1 and 2 depict perspective views of infusion system 100 for infusing insulin (or other fluid medicament or medication) into a user (patient) in pre-activation and post activation configurations, respectively. Infusion system 100 comprises a detachable activation mechanism 102 and device 104 (or pod) for delivering insulin to the user. (The other medicament (other than insulin) may be small molecule pharmaceutical solutions, large molecule or protein drug solutions, saline solutions, blood or other fluids known to those skilled in the art.) As described in more detail below, device 104 is a wearable apparatus, system or pod for diabetes management in which continuous glucose monitoring (CGM), insulin delivery and control functionality are provided to ensure insulin is delivered at very precise rates and has the capability of detecting occlusions in real time. In operation, device 104 is applied by opening a sterile packaging, filling the reservoir with insulin, priming the fluid path, removing the adhesive backing, sticking infusion system 100 to the desired body location, pushing a button assembly, removing and disposing the detachable activation mechanism 102. Removing detachable activation mechanism 102 components that are necessary for device 104 activation and needle insertion, but not required for infusion or sensing offers the benefit of a smaller, lower profile, more comfortable and discrete wearable device 104 for the user. This is described in more detail below.

In this example, detachable activation mechanism 102 is configured to deploy or apply device 104 onto the user for insulin delivery and to detach from the device 104 after deployment. In detail, detachable activation mechanism 102 is configured to insert an indwelling integrated infusion needle (or catheter introduced by an introducer needle) and CGM sensor (104-6 described below) of device 104 in a single insertion site (or more than one site) normal to a user's skin or at other desired angles known to those skilled in the art. Detachable activation mechanism 102 functions to insert the infusion needle (or catheter via an introducer needle) and/or CGM sensor via needle cartridge assembly 106 (also referred to as cartridge assembly 106). This is described in more detail below.

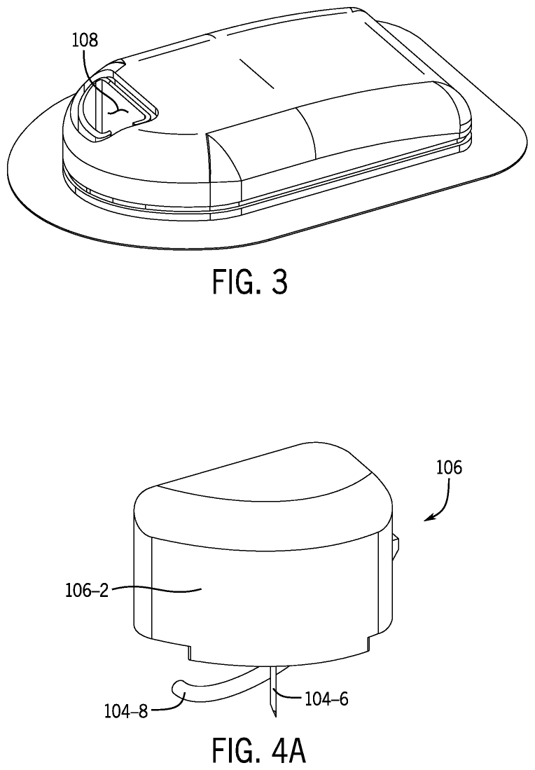

FIGS. 2 and 3 clearly show device 104, but FIG. 3 illustrates device 104 without needle cartridge assembly 106 (or cartridge assembly 106) shown. Infusion needle is preferably constructed of steel but it may be any rigid material known to those skilled in the art. In this embodiment described herein, detachable activation mechanism 102 and needle cartridge assembly 106 are described with respect to inserting an integrated infusion needle and CGM sensor. However, detachable activation mechanism 102 and/or cartridge assembly 106 may be used to insert an infusion needle or catheter only introduced by an introducer needle (without CGM sensor capability) as known to those skilled in the art. Further, detachable mechanism 102 and/or cartridge assembly 106 may be used to simultaneously insert an infusion catheter and a CGM sensor (or any other analyte sensor) separately via dual (separate) introducer needles. (An analyte sensor is designed to sense, detect or measure an analyte in a user as known to those skilled in the art.) This is described in more detail below.

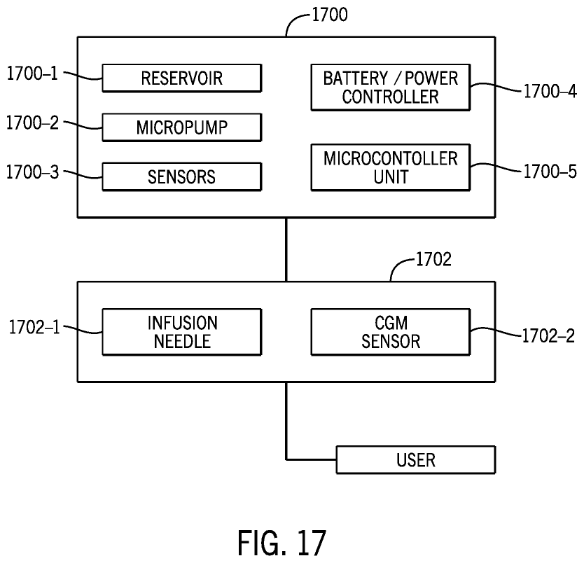

Device 104 incorporates, among other elements (as described below), a micropump (identified below with respect to FIG. 17) as known to those skilled in the art that can be used for pumping fluid, valves used for regulating flow, actuators used for moving or controlling the micropump and valves and/or sensors used for sensing pressure and/or flow. The micropump may be used to infuse the insulin or other fluidic medication to the user (patient). As indicated above, the medicament (medication) may include small molecule pharmaceutical solutions, large molecule or protein drug solutions, saline solutions, blood or other fluids known to those skilled in the art. Insulin is an example of fluid that is described in this application. However, micropump may be used in other environments known to those skilled in the art.

Device 104 also includes reservoir, a microcontroller unit (MCU) (FIG. 17), integrated infusion needle and CGM sensor 104-6 and a battery and power controller (FIG. 17). The reservoir is configured to receive and store insulin for its delivery over a course of about three days, or as needed. However, reservoir size may be configured for storing any quantity of fluid as required. The micropump fluidly communicates with reservoir to enable infusion as needed. CGM, as known to those skilled in the art, tracks patient glucose levels and permits those levels to be used in algorithms that control flow rate. MCU controls the operation of the micropump to deliver insulin through the insulin needle 104-6 from the reservoir at specific doses, i.e., flow rates over specified time intervals, based on CGM data converted to desired flow rate via control algorithms. The battery and power controller controls the power to the MCU and the micropump to enable those components to function properly as known to those skilled in the art. The CGM is powered by battery and the power controller through the MCU. As described above, a CGM sensor is one type of analyte sensor as known to those skilled in the art. However, device 104 may incorporate other types of analyte sensors for sensing or measuring an analyte in the user including keytones (as an example).

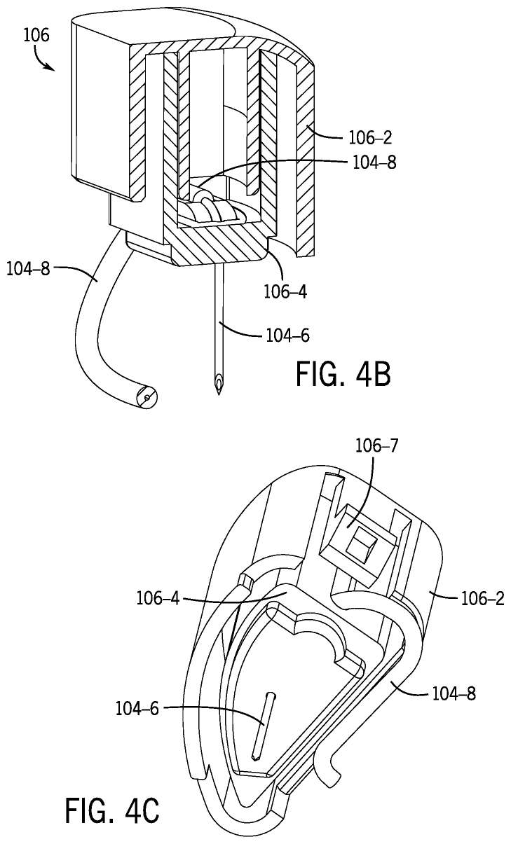

Infusion system 100 further incorporates needle cartridge assembly 106 as shown in FIGS. 4A-4D (cartridge assembly 106 comprising a housing or frame). Needle cartridge assembly 106 is configured to fit within a cartridge (insertion) compartment opening 108 in top housing 104-2 (described below) and a channel extending through top housing 104-2 of device 104 as shown in FIG. 3. The cartridge (insertion) compartment opening 108 in the top housing 104-2 enables access for the cartridge assembly 106 so it can drive integrated needle 104-6 into the subcutaneous tissue of a user.

Upon needle insertion, opening 108 is closed off, so the housing provides some sealing properties and creates a continuous surface as shown in FIG. 2. Detachable activation mechanism 102 compartment is outside of the hermetically sealed compartment that contains the pump and electronics of device 104 for delivering insulin so water ingress can occur without affecting device 104 performance. The amount of water ingress is limited by minimizing the volume of empty space in order to reduce the wetting nuisance that can occur after a user's swim or shower.

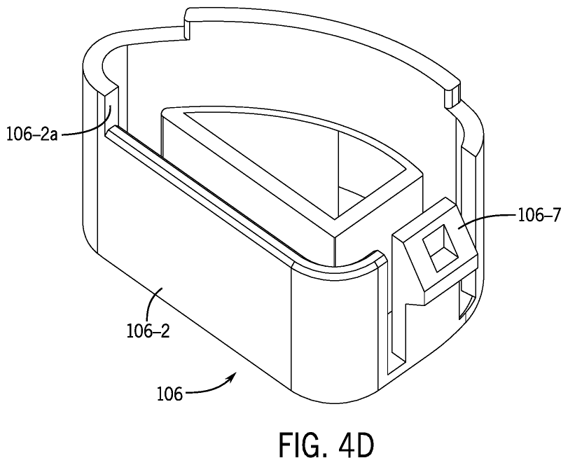

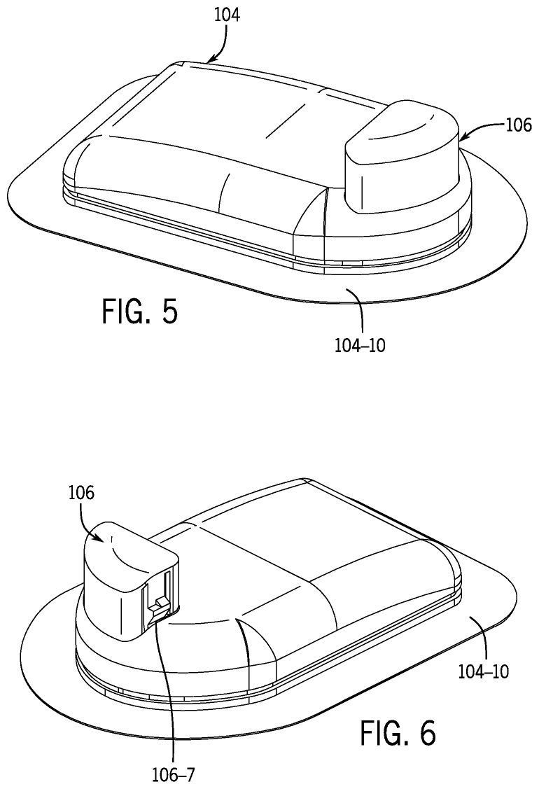

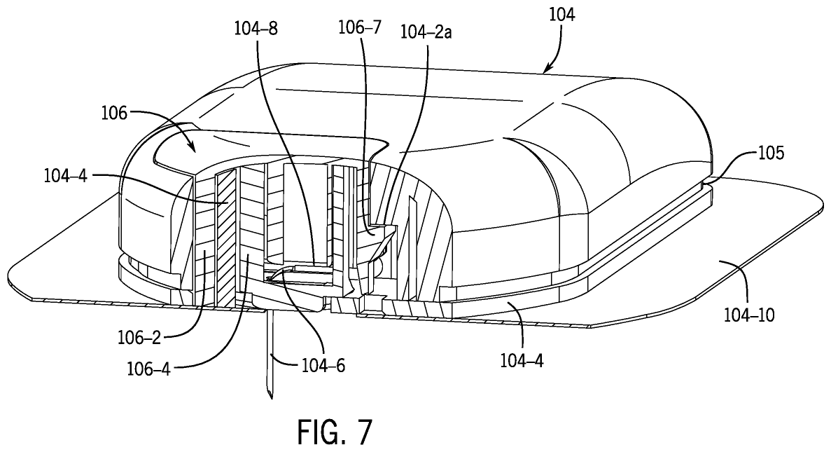

In brief, needle cartridge assembly 106 is configured to move from (1) a telescoping position above the top surface of device 104, as shown in FIGS. 5 and 6, wherein integrated infusion needle and CGM sensor 104-6 is in a retracted position to (2) an advanced position within device 104, as shown in FIGS. 2 and 7, wherein the infusion needle 104-6 is in a deployed position embedded into the subcutaneous layer of the user's tissue (after insertion). This is described in more detail below.

Device 104 for delivering insulin includes top housing 104-2, baseplate 104-4, integrated infusion needle and CGM sensor 104-6, insertion mechanism tubing 104-8 and adhesive patch 104-10. Device 104 as shown and described herein is configured to be mounted to the user (and remain on the user by way of adhesive patch 104-10). Device 104 includes groove 105 along the lower periphery thereof that is defined by housing 104-2 and baseplate 104-4 in an assembled configuration. Groove 105 is used to receive flanges of the components of detachable activation mechanism 102 as described in more detail below.

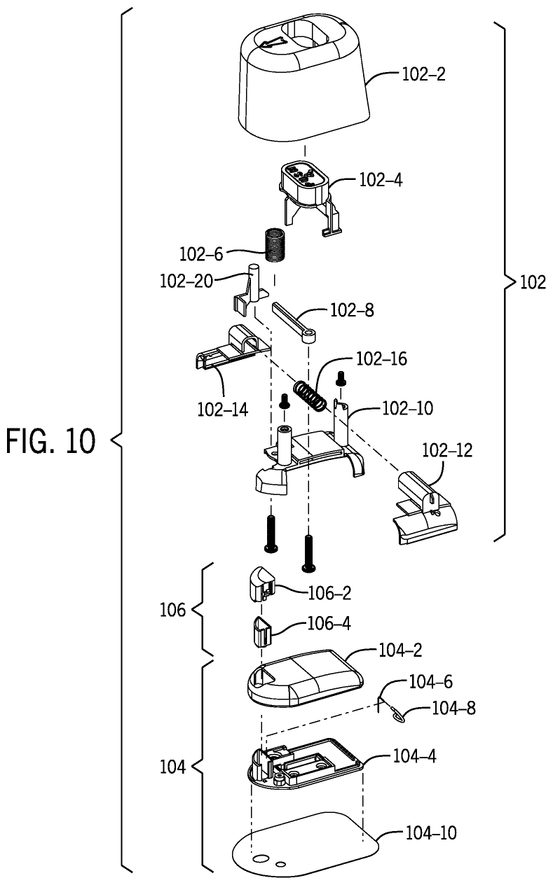

Detachable activation mechanism 102 includes detachable mechanism housing 102-2, activation button subassembly 102-4, insertion spring 102-6, activation beam 102-8, insertion mechanism inner housing 102-10, left release arm 102-12, right release arm 102-14, release spring 102-16, and a number of screws for assembling these components (shown but not numbered). These components are seen clearly in FIG. 10.

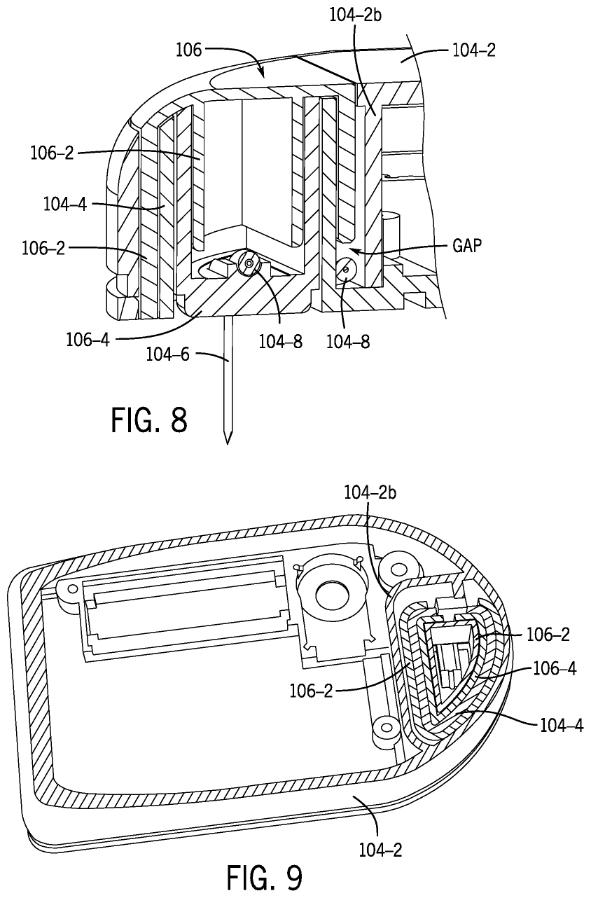

Needle cartridge assembly 106 includes needle cartridge 106-2, needle cartridge cover 106-4 and needle cartridge lock snap 106-7. Needle cartridges cover 106-4 slides within needle cartridge 106-2 and is fully integrated together as best shown in FIG. 4B. Integrated needle 104-6 is press fit or glued through an opening in needle cartridge cover 106-4 to extend beyond the bottom surface thereof. This is best shown in FIGS. 4B, 4C and FIG. 8. Tubing 104-8 is fluidly connected to integrated infusion needle and CGM sensor 104-6 as known to those skilled in the art and tubing 104-8 extends through side rear wall of cartridge cover 106-4 for fluid connection to a filled reservoir via a micropump. Lock snap 106-7 is configured to act as a locking mechanism to lock the cartridge assembly 106 down and force integrated infusion needle 104-6 in a user's skin. Needle cartridge lock snap 106-7 engages with a coupling ledge 104-2a as part of top housing 104-2 as seen in FIG. 7.

As indicated above, needle cartridge assembly 106 is configured to slide or move through a channel within device 104 from a telescoping position to a position fully integrated within device 104 whereby integrated infusion needle 104-6 is inserted into the subcutaneous tissue of the user. It is the construction of slidable needle cartridge assembly 106 and its operation with respect to device 104 offers the benefit of or facilitates a low profile configuration of device 104 while creating a hermetic seal to prevent leaks around the channel. To this end, needle cartridge assembly 106 is configured to slide through opening 108 and a channel defined by a wall in baseplate 104-4 and corresponding wall 104-2b in top housing 104-2 (FIG. 8 for example). Specifically, baseplate 104-4 includes a planar surface or floor (wall) and a wall extending perpendicular to that floor (wall). The perpendicular wall creates a compartment for receiving needle cartridge assembly 106. FIGS. 8 and 9 best illustrate the wall structure and interaction with needle cartridge assembly 106 and baseplate 104-4.

Needle cartridge assembly 106 is configured with dual spaced guide walls that are defined by needle cartridge 106-2 and needle cartridge cover 106-4 as fully assembled. Upon activation, perpendicular wall of baseplate 104-4 slides within the gap between dual spaced guide walls defined by needle cartridge 106-2 and needle cartridge cover 106-4. Note that the distal end of outer wall of needle cartridge 106-2 is configured as uneven in length (not constant), thereby creating a step section 106-2a (as best seen in FIG. 4D) in that wall or an opening section (shorter in length) or gap between the wall and baseplate 104-4 when needle cartridge assembly 106 is fully advanced into device 104. This gap ensures that this outer wall does not interfere or compress tubing 104-8 during operation. This is shown in FIG. 8.

As can be seen in FIG. 9, the configuration of wall 104-2b of top housing 104-2 that surrounds the dual walls of needle cartridge assembly 106 functions as a barrier to prevent any fluid seepage into remaining compartments of device 104 to protect other interior components such as a battery, power controller or MCU. That is, the wall structure (interaction and layering) of housing 104-2 in addition to the walls of needle cartridge assembly 106 as well as baseplate 104-4 help create a sealed compartment to function as the barrier to prevent fluid leakage into other compartments of device 104.

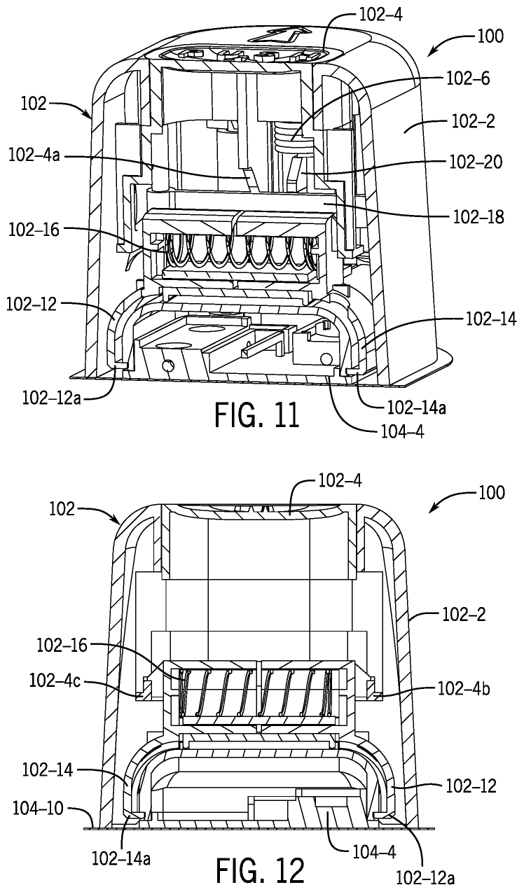

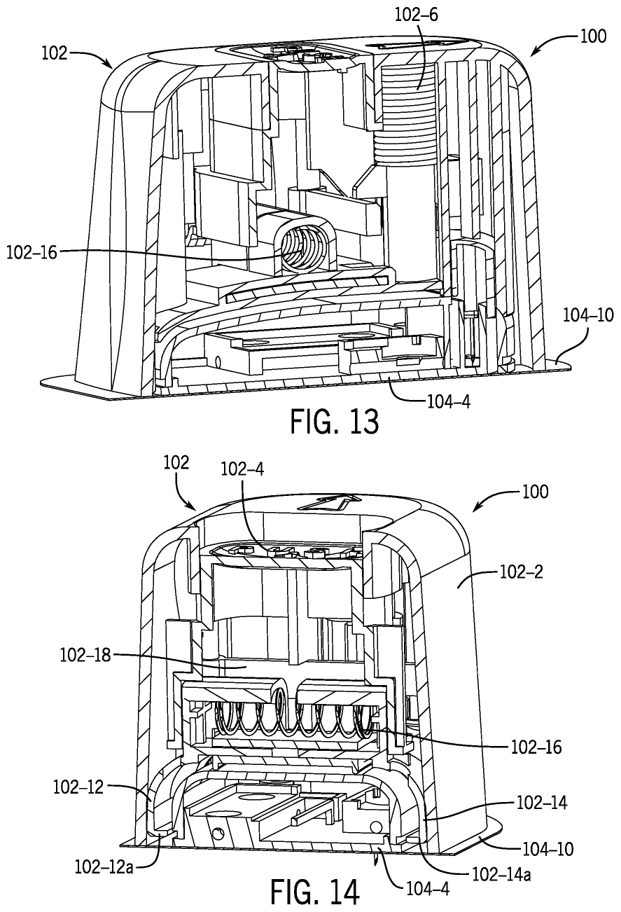

FIGS. 11-13 depict various cross-sectional views of the infusion system of FIG. 1 in a pre-activation configuration. In this configuration, device 104 for delivering insulin is securely attached to and within the detachable activation mechanism 102 by way of retention flanges 102-12a, 102-14a on right and left release arms 102-12, 102-14, respectfully. These flanges are biased outwardly by release spring 102-16, as shown in these FIGS. 11-13. In the locked position, flanges 102-12a, 102-14a engage groove 105 of device 104 to secure device 104 within detachable activation mechanism 102. Activation beam 102-8 holds insertion rod 102-20 in an upward position and spring 102-6 loaded. Button features or ledges 102-12b, 102-14b lock the release arms 102-12, 102-14 in the closed position so that flanges 102-12a, 102-14a within groove 105 lock the device 104 in detachable activation mechanism 102. Button ledges 102-12b, 102-14b interfere with release arms 102-12, 102-14 before activation.

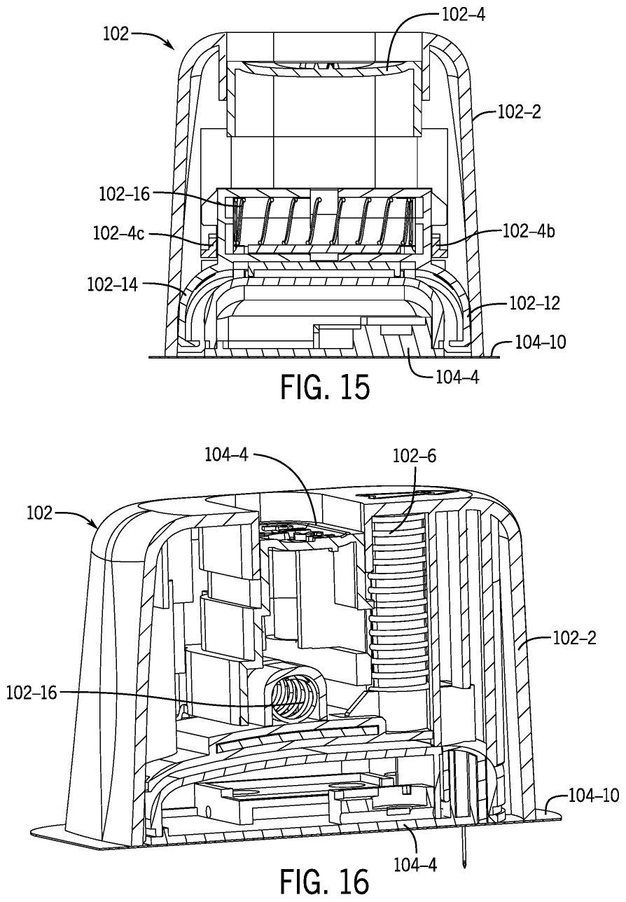

When activation button assembly 102-4 is pressed, the ramped surface 102-4a on button assembly 102-4 translates downward, causing the activation beam 102-8 to rotate, freeing the release of insertion rod 102-20. Under load from spring 102-6, insertion rod 102-20 moves downward, thereby forcing cartridge assembly 106 downward as well. Insertion rod 102-20 pushes infusion needle 104-6 into an inserted position within the subcutaneous tissue of the user. Simultaneously, when button assembly 102-4 translates downward by force from the user, button ledges 102-4b, 102-4c of button assembly 102-4 (of detachable activation mechanism 102) move into a position without interference from the release arms and spring 102-16 causes ribs 102-12a, 102-14b of release arms 102-12, 102-14 to move outwardly, thereby moving flanges out of a groove 105 (also called indentation), releasing device 104. FIGS. 14-16 depict various cross-sectional views of the infusion system of FIG. 1 in a post activation configuration, but before detachable actuation mechanism 102 has been removed. Retention flanges 102-12a, 102-14a have moved outwardly, allowing device 104 to detach from detachable activation mechanism 102.

FIG. 17 depicts a block diagram of example components of (1) device 1700 for delivering insulin and (2) cartridge assembly 1702 of infusion system 100 as described in detail above. (Device 104 and cartridge assembly 106 are renumbered as device 1700 and cartridge assembly 1702 in FIG. 17.) Specifically, device 1700 incorporates several components or modules (not shown) in the fluidic pathway including reservoir 1700-1 for storing the insulin, micropump 1700-2 for pumping the insulin, sensors 1700-3 (e.g., pressure) for sensing various parameters in the system and user and tubing connecting infusion needle 1702-1 to reservoir 1700-1 within cartridge assembly 1702. Device 1700 also includes microcontroller unit (MCU) 1700-4 and battery and power controller 1700-5. Cartridge assembly 1702 also includes CGM sensor 1700-5. CGM, as known to those skilled in the art, tracks user glucose levels and permits those levels to be used in algorithms that control flow rate. MCU 1700-4 controls the operation of micropump 1700-2. Infusion needle 1702-1 and CGM sensor 1702-2 are shown as separate components in FIG. 17 for illustration purposes. Infusion needle 1702-1 and CGM sensor 1702-2 may be integrated or cartridge assembly 1702 may only include infusion needle 1702-1 or CGM sensor individually (without the other).

Reservoir 1700-1 is configured to receive and store insulin for its delivery over a course of about three days, or as needed. However, reservoir size may be configured for storing any quantity of fluid as required.

MCU 1700-5 electronically communicates with sensors 1700-3 and micropump 1700-2 as well as the CGM sensor 1702-2, as the monitoring components. Among several functions, MCU 1700-5 operates to control the operation of micropump 1700-2 to deliver insulin through insulin needle 1702-1 from reservoir 1700-1 at specific doses, i.e., flow rates over specified time intervals, based on CGM data converted to desired flow rate via control algorithms.

Battery and power controller 1700-4 controls the power to MCU 1700-5 and micropump 1700-2 to enable those components to function properly as known to those skilled in the art. CGM sensor 1700-2 is powered by battery and power controller 1700-4 through MCU 1700-5.

The components of device 1700 and cartridge assembly 1702 shown in FIG. 17 are only a few components. Those skilled in the art know that device 1700 and cartridge assembly 1702 include additional components.

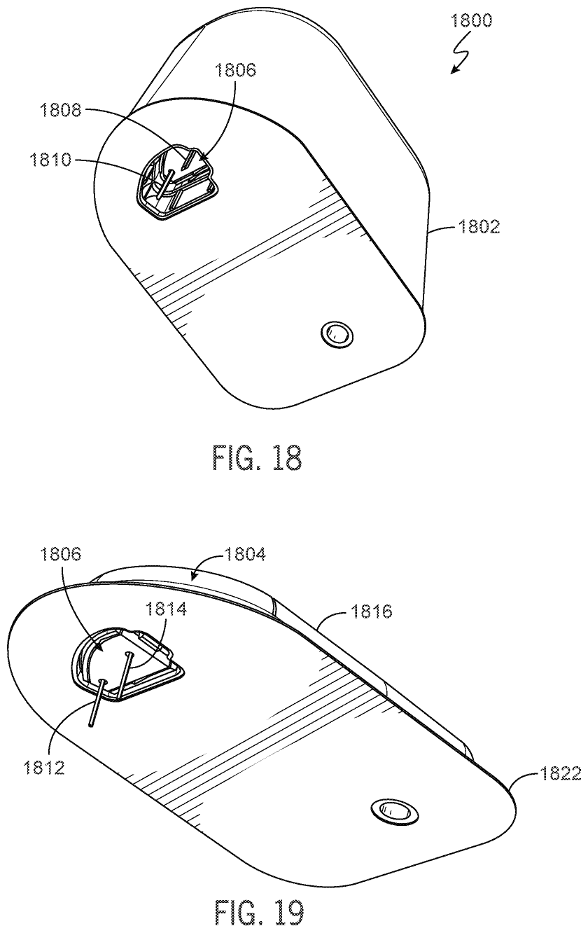

FIGS. 18 and 19 depict bottom perspective views of infusion system 1800 for infusing insulin (or other fluid medicament or medication) into a user (patient) in pre-activation and post activation configurations, respectively. Similar to the above example, infusion system 1800 comprises detachable activation mechanism 1802 and device 1804 (or pod) for delivering insulin (or other medicament) to the user. Similar to the above, device 1804 is a wearable apparatus, system or pod for diabetes management in which continuous glucose monitoring (CGM), insulin delivery and control functionality are provided to ensure insulin is delivered at very precise rates and has the capability of detecting occlusions in real time. The compartment of detachable activation mechanism 1802 is outside of the hermetically sealed compartment of device 1804 that contains a pump and electronics so water ingress can occur without affecting device performance. This is described in more detail below.

In brief, device 1804 is preassembled in a single use detachable activation mechanism 1802. Upon activation as similar to the above example, detachable activation mechanism 1802 releases a spring (FIG. 28) which drives introducer needles 1808, 1810 coupled with (carrying) infusion catheter 1812 and continuous glucose monitoring (CGM) sensor 1814, respectively into the skin. (CGM sensor 1814 is one example of an analyte sensor). Detachable activation mechanism 1804 passively detaches upon activation. The spring, activation and release components for releasing device 1802 are thereby removed when detachable activation mechanism 1804 is removed and disposed in the trash. (Similar to the example device for delivering insulin above, CGM sensor 1814 is employed, but those skilled in the art know that other analyte sensors may be used to achieved desired results as described herein.) Introducer needles 1808, 1810 remain attached to the device 1804 (pod) and are removed by the user using a hub 1807 (described below).

In a bit more detail, device 1804 is applied by opening a sterile packaging, filling the reservoir with insulin, priming the fluid path, removing the adhesive backing, sticking infusion system 1800 to the desired body location, pushing a button assembly, removing and disposing the detachable activation mechanism 1802. Removing detachable activation mechanism 1802 components that are necessary for device 1804 activation and introducer needles insertion, but not required for actual infusion or sensing offers the benefit of a smaller, lower profile, more comfortable and discrete wearable device 1804 for the user. This is described in more detail below.

Detachable activation mechanism 1802 is configured to insert infusion catheter 1812 and CGM sensor 1814 of device 1804, simultaneously, at an insertion site normal to a user's skin or at other desired angles known to those skilled in the art. In this embodiment, specifically, detachable activation mechanism 1802 and cartridge assembly 1806 are described with respect to inserting two separate introducer needles 1808, 1810 as described below to insert an infusion catheter 1812 and CGM sensor 1814 (or other analyte sensor) simultaneously. Introducer needles 1808, 1810 are preferably constructed of steel, but the needles may be any rigid material known to those skilled in the art.

Device 1804 incorporates, among other elements (as described herein and as shown in FIG. 17, a micropump as known to those skilled in the art that can be used for pumping fluid, valves used for regulating flow, actuators used for moving or controlling the micropump and valves and/or sensors used for sensing pressure and/or flow. The micropump may be used to infuse the insulin or other fluidic medicament or medication to the user (patient). As described above, medicament (medication) may include small molecule pharmaceutical solutions, large molecule or protein drug solutions, saline solutions, blood or other fluids known to those skilled in the art. Insulin is an example of fluid that is described in this application. However, micropump may be used in other environments known to those skilled in the art. In addition, device 1804 may employ other kinds of pumps (other than micropumps).

Device 1804 also includes a reservoir, a microcontroller unit (MCU) (FIG. 17), infusion catheter and CGM sensor 1814 and a battery and power controller (FIG. 17). As described above, the reservoir is configured to receive and store insulin (or other medicament) for its delivery over a course of about three days, or as needed. However, reservoir size may be configured for storing any quantity of fluid as required. The micropump fluidly communicates with reservoir to enable infusion as needed. As described above, CGM, as known to those skilled in the art, tracks patient glucose levels and permits those levels to be used in algorithms that control flow rate. MCU controls the operation of the micropump to deliver insulin through the insulin catheter 1812 from the reservoir at specific doses, i.e., flow rates over specified time intervals, based on CGM data converted to desired flow rate via control algorithms. The battery and power controller control the power to the MCU and the micropump to enable those components to function properly as known to those skilled in the art. The CGM is powered by battery and the power controller through the MCU.

Infusion system 1800 further incorporates cartridge assembly 1806 as shown in FIGS. 18-33 along with introducer needle hub 1807 extending from cartridge assembly 1806. Cartridge assembly 1806 has a housing and it is configured to fit within a cartridge assembly (insertion) compartment opening 1809 in top housing 1816 (described below) and into a channel extending through top housing 1816 of device 1804. The housing may include a ledge to function as a stop, thereby prevent cartridge assembly 1806 from sliding through the channel or extending beyond the baseplate 1818. The cartridge assembly (insertion) compartment opening 1809 in the top housing 1816 enables access for the cartridge assembly 1806 so it can drive introducer needles 1808, 1810 into the subcutaneous tissue of a user. Introducer needle hub 1807 is configured to be removable and function to withdraw introducer needles 1808, 1810 as described in more detail below.

Upon needle(s) insertion, opening 1809 is closed off, so the housing provides some sealing properties and creates a continuous surface as shown in FIG. 19 for example. As described above herein, detachable activation mechanism 1802 compartment is outside of the hermetically sealed compartment that contains the pump and electronics of device 1804 for delivering insulin so water ingress can occur without affecting device 1804 performance. The amount of water ingress is limited by minimizing the volume of empty space in order to reduce the wetting nuisance that can occur after a user's swim or shower.

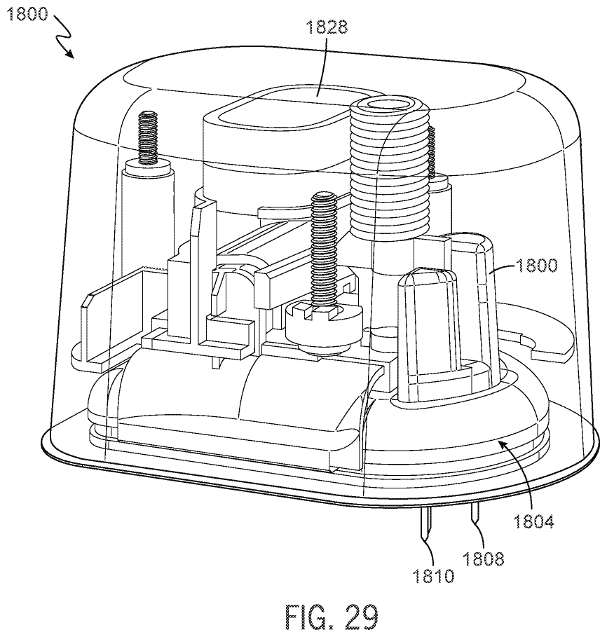

In brief, needle cartridge assembly 1806 is configured to move from (1) a telescoping position above the top surface of housing 1816 of device 1804 as shown in FIG. 28 for example, wherein infusion catheter 1812 and CGM sensor 1814 are in a retracted position within device 1804 to (2) an advanced position within device 1804, as shown in FIG. 29-33 for example, wherein the infusion catheter 1812 and CGM sensor 1814 are in a deployed position embedded into the subcutaneous layer of the user's tissue (after insertion). While this is described in more detail below, infusion catheter 1812 and CGM sensor 1814 are simultaneously inserted into the subcutaneous tissue layer of a user for wearable insulin infusion delivery device 1804 that includes a continuous glucose monitor (CGM).

Device 1804 may be a closed loop or partially closed loop wearable insulin delivery device driven by blood glucose sensing feedback. Integrating these two capabilities into a single wearable device provides a more discreet and convenient means of optimizing blood glucose levels compared to the currently available commercial options. Combining insertion for CGM and infusion into a shared mechanism also has the advantages of reducing the number of insertion events users must endure. (Device 1804 may alternatively be an open loop wearable insulin delivery device whereby the user will control the insulin flow without integration between the sensor and pump.)

Device 1804 for delivering insulin includes top housing 1816, baseplate 1818, introducer needles 1808, 1810, infusion catheter 1812 and CGM sensor 1814, insertion mechanism tubing 1820 and adhesive patch 1822. Device 1804 includes groove 1824 along the lower periphery thereof that is defined by housing 1816 and baseplate 1818 in an assembled configuration. Groove 1824 is used to receive flanges of the components of detachable activation mechanism 1802 as described in detail below (and above with respect to the example device 104).

Similar to the structure and functionality of example infusion system 100 above with respect to FIGS. 1-17, two release arms are configured to move from a position wherein the device is secured within detachable activation mechanism 1802 and a second position where the device is released from detachable activation mechanism 1802 upon activation by the user. The two release arms each include a flange that fits within groove 1824, thereby securing the device within detachable activation mechanism 1802. A spring (shown in FIG. 28 for example) is used to bias the two arms outwardly, releasing the flanges and thereby releasing the device from the detachable activation.

In the embodiments described herein, infusion catheter 1812 may be a PEEK infusion catheter or made of other materials with suitable biocompatibility, strength, flexibility and insulin compatibility, connected directly to the insulin fluid path that is inserted with introducer needle 1808 that is preferably U-shaped. Other catheter materials and insertion designs are possible with mechanism 1802 including an untipped stainless-steel catheter inserted with a U-shaped introducer needle and a standard Teflon catheter inserted with a hypodermic needle. Introducer needle 1810 is also preferably U-shaped, but it may be any shape as known to those skilled in the art.

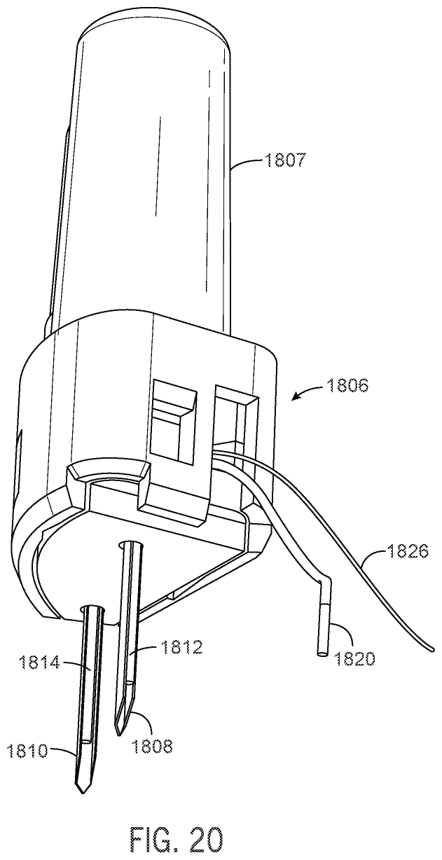

FIG. 20 depicts cartridge assembly 1806 with infusion catheter 1812 and CGM sensor 1814. Cartridge assembly 1806 is connected to an infusion fluid path and CGM electronics, as described below, and assembled into device 1804 before being assembled into detachable activation mechanism 1802. These are the components driven downwardly by the detachable activation mechanism 1802 to insert infusion catheter 1812 and CGM sensor 1814. Infusion catheter tubing 1820 extends from infusion catheter 1812 through a window in cartridge assembly 1806 as shown. CGM connection wires 1826 are connected to CGM sensor 1814 and also extend through the same window as shown.

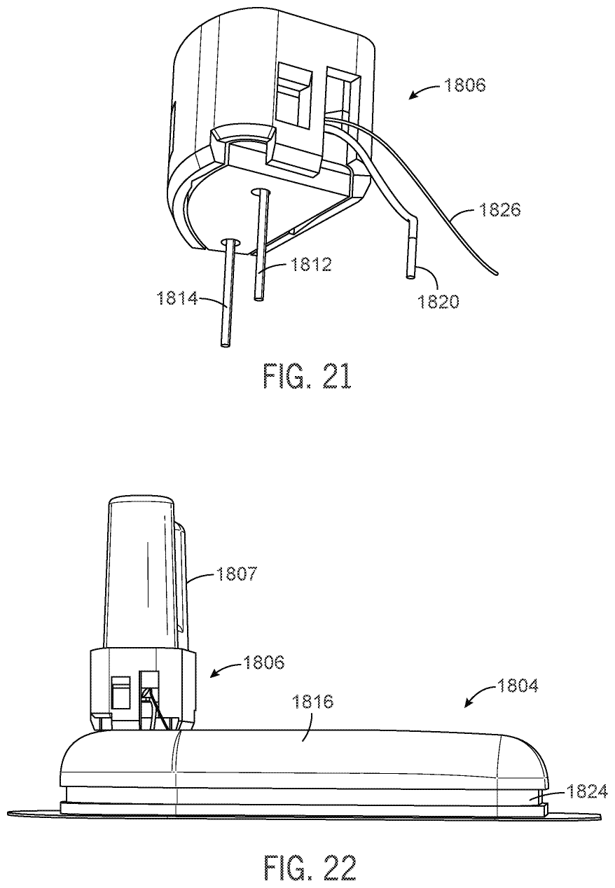

FIG. 21 depicts cartridge assembly 1806 with introducer needles 1808, 1810 removed to illustrate the configuration after insertion and during use.

FIG. 22 depicts cartridge assembly 1806 fluidically and electrically connected to the device 1804. This is the configuration of device 1804 before it is assembled into detachable activation mechanism 1802. In this configuration, introducer needles 1808, 1810 are coupled with (carrying) infusion catheter 1812 and CGM sensor 1814 and cartridge subassembly 1806 is in the upward pre-activation position (subcutaneous). In one embodiment, a PEEK infusion catheter is employed and the advantage of a PEEK infusion catheter is that it can connect directly to the insulin fluid path septum without the need for additional intermediate fluidic sealing components.

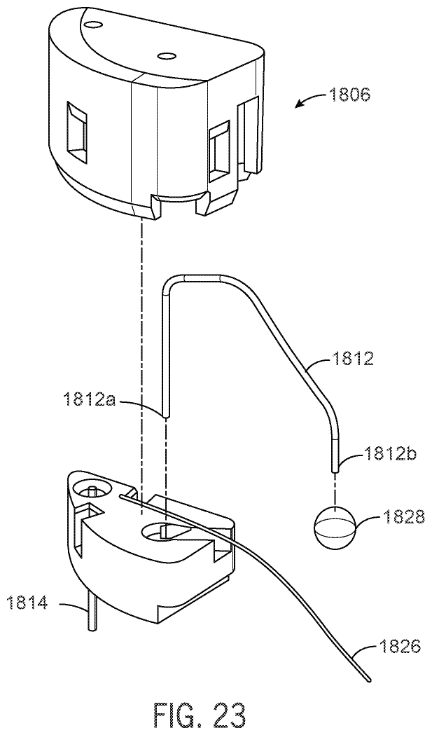

FIG. 23 depicts an exploded view of cartridge assembly 1806 that may be installed in device 1804 in pre-activation configuration. Infusion catheter 1812 has user (patient) end 1812a and septum connecting end 1812b of infusion catheter 1812. Several components are shown including fluid path ball septum 1828.

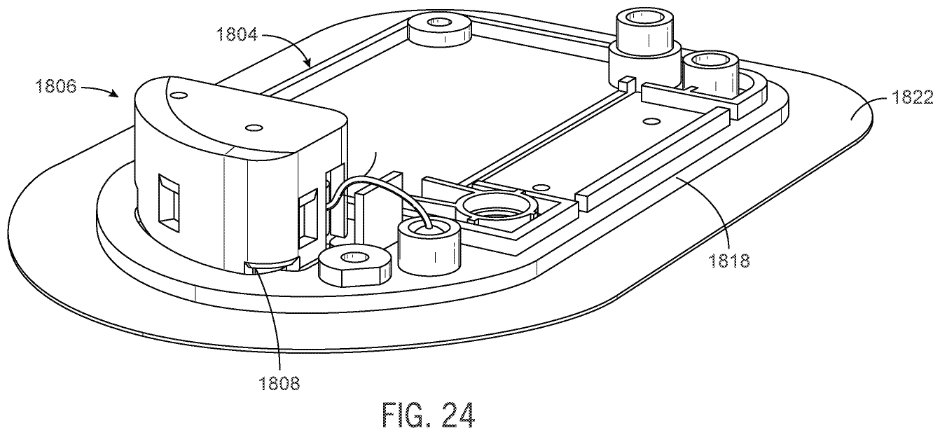

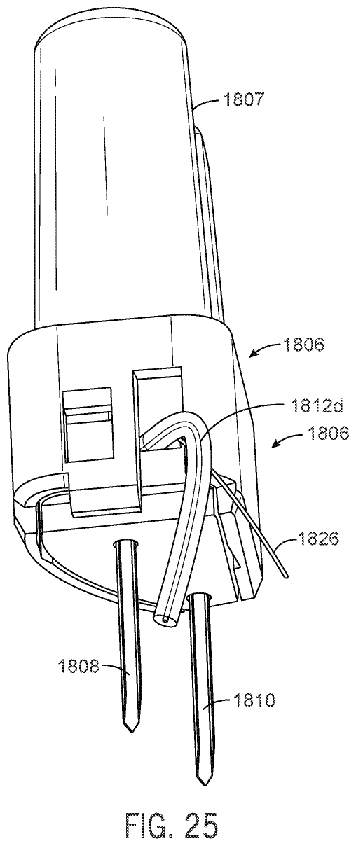

FIG. 24 depicts a portion of device 1804 exposing inner components. Infusion catheter connection to the fluid path port in baseplate 1818 is shown. Alternately, infusion catheter 1812 can be comprised of an untipped (dull) stainless steel hypodermic tubing 1812c connected to the fluid path with flexible plastic tubing 1812d as shown in FIG. 25. Tubing 1812c would be inserted by a U-shaped introducer needle 1808.

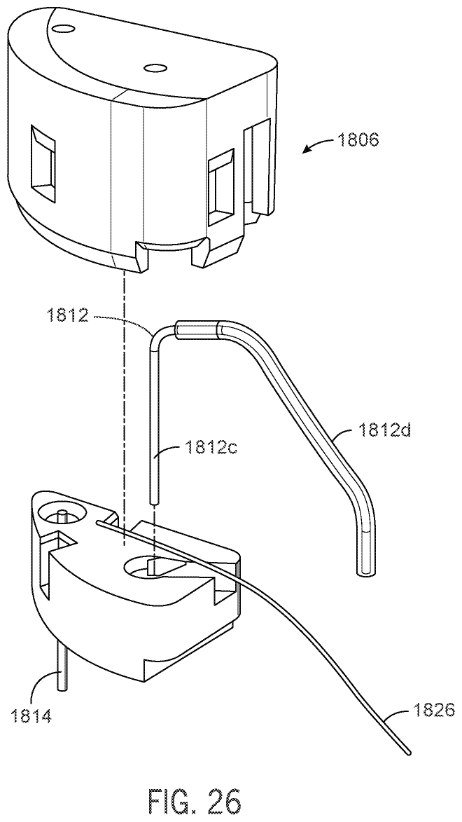

FIG. 26 depicts an exploded view of cartridge assembly 1806 shown in FIG. 25 with CGM connection wires 1826 but without hub 1807.

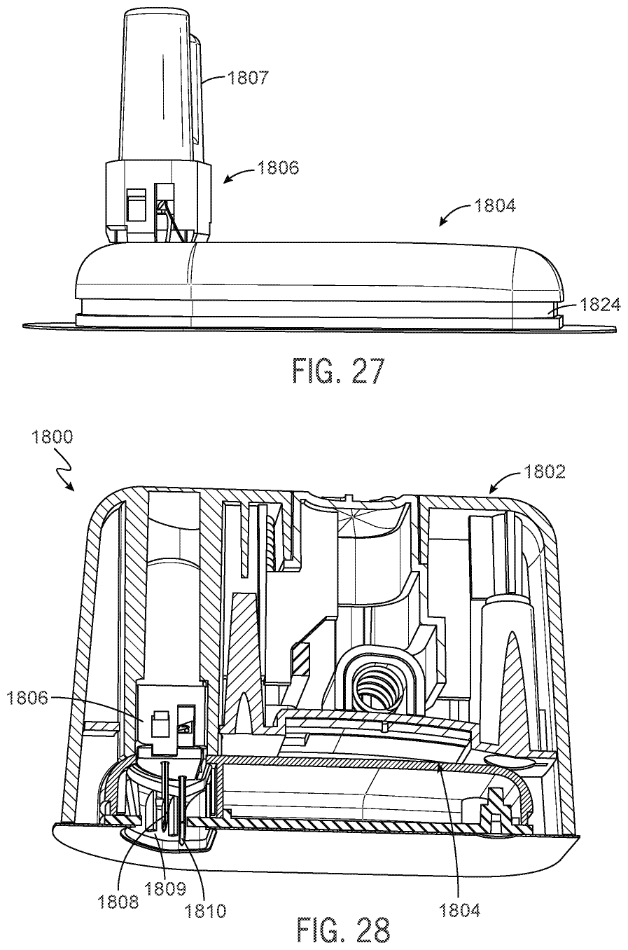

FIG. 27 depicts a view of cartridge assembly 1806 installed within device 1804 but before it is assembled into detachable activation mechanism 1802.

FIG. 28 depicts a view of infusion system 1800 in which device 1804 is in a pre-activation configuration that is assembled into detachable activation mechanism 1802. Cartridge assembly 1806 is in an upward position. The springs for introducer needle insertion and device 1804 release are loaded as shown. (The introducer spring is not shown and device 1804 release spring is shown in FIG. 28. This springs are the same the example device 104 in FIGS. 1-17.)

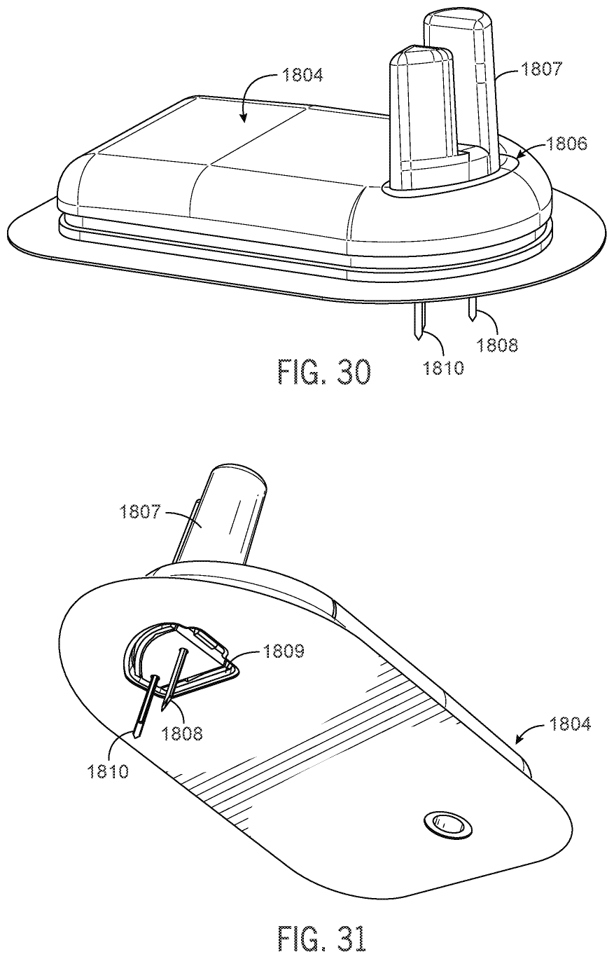

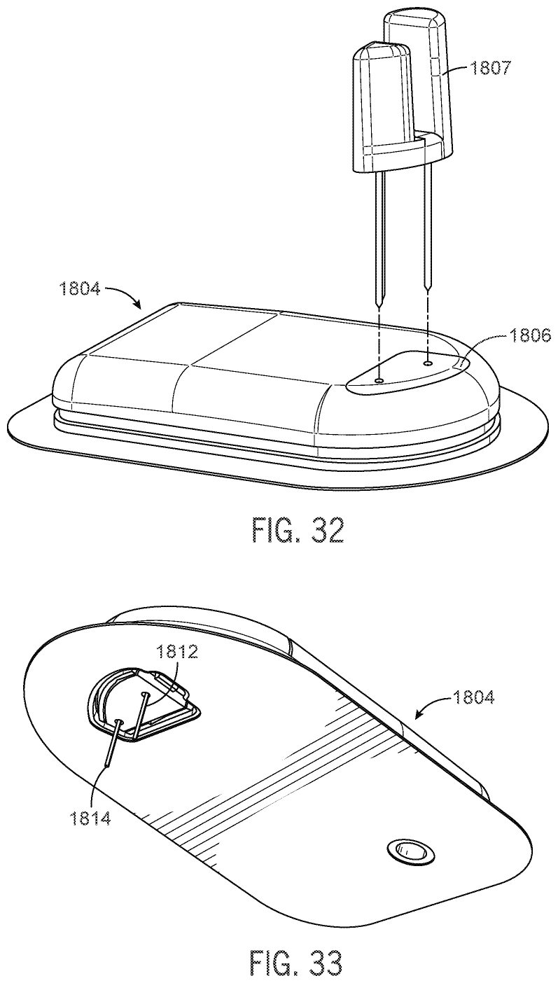

FIG. 29 depicts a transparent view of infusion system 1800 in which button 1828 has been pressed which rotates a release beam allowing an insertion rod to drive introducer needles 1808, 1810 into a user's tissue. Introducer needles 1808, 1810 remain in the tissue. After activation, the user removes detachable activation mechanism 1802 leaving device 1804 as shown in FIGS. 30 and 31. Insertion needle hub 1807 protrudes from device 1804 and is used as a handle to manually pull-out (withdraw) introducer needles 1808, 1810. This is shown in FIG. 32. FIG. 33 depicts a view of device 1804 with introducer needles 1808, 1810 removed.

It is to be understood that the disclosure teaches examples of the illustrative embodiments and that many variations of the invention can easily be devised by those skilled in the art after reading this disclosure and that the scope of the present invention is to be determined by the claims below.

What is claimed is:

1. An infusion system comprising:
a device for delivering medicament to a user, the device configured to be mounted on the user;
an infusion needle for delivering the medicament to the user; and
a needle cartridge assembly for supporting the infusion needle, the needle cartridge assembly configured to move from (1) a telescoping position above the device for delivering medicament, wherein the infusion needle is in a retracted position within the device for delivering medicament to (2) an advanced position within the device, wherein the infusion needle is in a deployed position inserted into a subcutaneous tissue of the user's skin.

2. The infusion system of claim 1 further comprising an analyte sensor for measuring an analyte in the user that is supported by and configured to move along with the cartridge assembly from the telescoping position, wherein the analyte sensor is in the retracted position within the device for delivering medicament to the advanced position, wherein the infusion needle is in a deployed position inserted into a subcutaneous tissue of the user's skin.

3. The infusion system of claim 2 wherein the analyte sensor and infusion needle are integrated.

4. The infusion system of claim 1 further comprising a detachable activation mechanism for (a) moving the needle cartridge assembly from the telescoping position to the advanced position and for (b) releasing the device for delivering medicament upon activation of the detachable activation by the user.

5. The infusion system of claim 4 wherein the detachable activation mechanism includes first and second release arms that are configured to move from a first position wherein the device is secured within the detachable activation mechanism to a second position wherein the device is released from the detachable activation mechanism upon activation by the user.

6. The infusion system of claim 5 wherein the first and second arms each include a flange for engaging the device for delivering medicament to the user, thereby securing the device within the detachable activation mechanism.

7. The infusion system of claim 6 wherein the detachable activation mechanism includes a spring for biasing the first and second arms outwardly.

8. The infusion system of claim 6 wherein the device for delivering includes a groove for (a) receiving the flanges when the first and second arms are in the first position, thereby securing the device for delivering medicament within the detachable activation mechanism and for (b) enabling the release of the flanges when the first and second arms are in the second position, thereby releasing the device from the detachable activation.

9. The infusion system of claim 1 wherein the analyte sensor is a CGM sensor for measuring glucose level in the user and the medicament is insulin.

10. An infusion system comprising:
a device for delivering medicament to a user, the device configured to be mounted on the user;
a needle for infusing medicament into the user or introducing an infusion catheter to infuse the medicament into the user;
a needle cartridge assembly for supporting the needle, the needle cartridge assembly configured to move from (1) a telescoping position above the device for delivering medicament, wherein the needle is in a retracted position within the device for delivering medicament to (2) an advanced position within the device, wherein the needle is in a deployed position inserted into a tissue of the user's skin.

11. The infusion system of claim 10 further comprising an analyte sensor for measuring an analyte in the user that is supported by and configured to move along with the cartridge assembly from the telescoping position, wherein the analyte sensor is in the retracted position within the device for delivering medicament to the advanced position, wherein the needle is in a deployed position inserted into the tissue of the user.

12. The infusion system of claim 10 wherein the analyte sensor and needle are integrated.

13. The infusion system of claim 10 wherein the medicament is insulin.
